(19) United States
(12) Patent Application Publication
(10) Pub. No.: US 2006/0172437 A1
(43) Pub. Date: Aug. 3, 2006

(54) Method for manufacturing a carrier for chemical or biochemical assays

(75) Inventor: Wouter Cornelis Puijk, Lelystad (NL)

Correspondence Address: MICHAELSON AND WALLACE;PARKWAY 109 OFFICE CENTER, 328 NEWMAN SPRINGS RD, P O BOX 8489, RED BANK, NJ, 07701, US

(73) Assignee: Pepscan Systems B.V.

(21) Appl. No.: 11/391,842

(22) Filed: Mar. 29, 2006

Related U.S. Application Data
(63) Continuation of application No. 09/744,230, filed on Mar. 21, 2001, now abandoned.

(30) Foreign Application Priority Data
Jul. 21, 1998 (NL) 1009703

(51) Int. Cl. G01N 33/543; B05D 3/02

(52) U.S. Cl. 436/518; 427/2.11

(57) ABSTRACT

Method for manufacturing a preparation carrier, in particular suitable for use in chemical and biochemical research, wherein: on at least one surface of a carrier base, a layer of plastic is provided, wherein the plastic layer is treated thermally and/or chemically, such that the surface roughness of the side of the plastic that faces the carrier base is reduced, while it does not adhere to the carrier base, whereupon the plastic is removed from the carrier base, with the released, relatively smooth surface of the plastic forming a carrier surface.

The invention relates to a method for manufacturing a preparation carrier, in particular suitable for use in chemical and biochemical research.

In biochemical research, use is typically made of so-called miniwells in for instance microtiter plates, wherein into each miniwell, a small amount of preparation to be assayed is introduced, treated and observed. By means of markers, it can then be established whether particular bindings have taken place in the relevant miniwells, whereby the nature of the preparation to be examined can be determined.

Such method has the advantage that a uniform distribution of the preparation can be obtained, as a result of which different assays can be performed simultaneously on the same preparation and/or the same assays can be performed on different preparations. However, such method has the drawback that the minimum volume of a miniwell is relatively large, for instance about 3 microliter, which means that relatively much preparation is required for performing the different assays, while, moreover, only a limited number of microwells can be provided on a specific surface. This means that such a method requires relatively much space on a preparation carrier.

There is further known a method wherein use is made of pins on which a preparation to be assayed is provided, which pins can subsequently be dipped in fluids included in the well of a microtiter plate, such that bindings may or may not take place between the preparation to be assayed and the fluids in the different wells. Such a method, too, has the drawback that for a relatively small number of preparation parts to be examined, a preparation carrier having a relatively large surface is required.

The microtiter plates and pins, used in the above method, can be manufactured from plastic, for instance polyethene, which plastic may or may not be provided with a reactive substance, such that specific bindings thereto are possible. The plastic used has a relatively slight flatness. The local flatness is considerably less than the local flatness of, for instance, a glass or mica surface. In this context, ‘local flatness’ should be understood to mean flatness of a relatively small surface, for instance in the order of square micrometers. This means that elements from the preparation bound thereto, provided with a marker, are relatively difficult to perceive, in particular because a microscope or photographic apparatus to be employed for the analysis thereof cannot be properly focused thereon. Indeed, due to the relatively high roughness of the surface on which the elements are bound, these elements will be staggered relative to each other, viewed in a direction at right angles to the relevant surface, which complicates focusing thereon. This means that the frontal surface of each well or pin to be analyzed should be relatively large to have sufficient distinctiveness. This impedes further scaling down.

Wang et al.: “Atomic Force Microscopy Study of Latex Film Formation” discloses the preparation of films by pouring a few drops of latex dispersion onto a freshly cleaved mica surface and allowing the film to dry at 36° C. for four hours. Then atomic force microscopy images are reported for the surfaces of said latex films. These films often show a highly ordered surface structure.

The latex film under investigation stays in full contact with the mica surface, the other side of said film being investigated during a prolonged time. In a method according to this publication the normal surface structure of latex film and changes therein is the subject of investigation. The surface roughness as disclosed in Wang at al. is however on a level of 10% or more (Z-axis/X-axis*100%), which is comparable to normally used polyethylene film.

U.S. Pat. No. 5,627,079 discloses a method for preparation of fluorinated or fluor containing surfaces, for example in order to refuntionalized said surface, for example for binding enzymes, antibodies and peptides, useful in the fabrication of biological sensors. Several methods are described for preparing such fluorinated surfaces, starting from all kinds of different materials. Essential in a product according to this publication is said (oxy)fluorinated surface. No indication is given of surface smoothness or flatness.

GB 471882 discloses a method for manufacturing artificial glass products having smooth surfaces by manufacturing in a mould and high polymerisation of the said surfaces.

The object of the invention is to provide a method of the type described in the preamble, in which the drawbacks mentioned of the known methods are avoided, while the advantages thereof are maintained. To that end, a method according to the invention is characterized by the features of claim 1.

The advantage achieved by providing a preparation carrier having a particularly flat plastic carrier surface, suitable for binding the desired elements in a preparation, is that elements that are to be detected particularly close together can be bound while they can nevertheless be distinguished from one another by, for instance, a microscope or a CCD-camera or a like apparatus.

In principle, plastic is a favorable material for manufacturing preparation carriers, in that it is relatively simple to process and is relatively strong, while a proper binding thereto of different preparations, in particular biochemical preparations such as viruses, antigens, peptides and the like, can be effected.

Surprisingly, it has now been found that by a method according to the present invention, a smooth plastic surface can be obtained such that it is actually suitable, or at least much better suitable, as carrier surface for preparations in such examination. Indeed, by forming the plastic layer, treated thermally or chemically, against a surface of a carrier base with a suitable surface roughness, it appears that the surface roughness of the surface lying against the carrier base can thereby be reduced considerably. Thus, for instance, a reduction of the surface roughness by a factor of 5-20 or more can be realized. This means that elements of a preparation that are bound to the carrier surface can have particularly small dimensions, while the presence thereof can nevertheless be optimally established therewith on the basis of, for instance, markers bound thereto. On a small carrier surface obtained by a method according to the invention, many different or identical elements can be distinguished close together. This can for instance be effected by applying drops of from 0.25 to 0.5 nl to the surface. In a preferred embodiment, these drops are applied by a printer, in particular a printer of the inkjet or bubblejet type or a like, preferably piezoelectrically controlled printer. Such printers are known per se. The use thereof for manufacturing (bio)chemical preparations is particularly advantageous in that a precise positioning and dosing can be obtained at high speed and reproducibility.

Moreover, particularly small wells can also be filled thereby, for instance in the order of magnitude of 0-3 μl, more in particular between 0 and 0.1 μl. Preferably, in a method according to the invention, such wells have said reduced surface roughness, yet in assays utilizing, for instance, fluorescence markers or the like, the inner surface of the wells may also be of rougher design, for instance of the normal roughness of PE.

In a particularly advantageous embodiment, a method according to the invention is characterized by the features of claim 2.

By at least partially melting the plastic against a surface of the carrier base, an optimal distribution of the plastic can be effected in a particularly simple manner. Moreover, in that case, for instance plastic film or sheet can readily be started from. However, it is also possible to cause for instance polymerization of the plastic layer to take place on the carrier surface, or to chemically treat the plastic such that deliquescence against the surface of the carrier base occurs.

Without wishing to be bound to any theory, the particular smoothness of the obtained carrier surface seems to result at least partly from the use of a particularly smooth carrier base and the absence of adhesion to the carrier base. Hence, it seems that a method according to the present invention can be optimized by using a carrier base having an optimal smoothness and the absence of adhesion between the plastic and the carrier base. However, also with sub-optimal conditions, sufficiently smooth carrier surfaces can already be obtained.

In a first preferred embodiment, a method according to the invention is further characterized by the features of claim 3.

The use of a plastic having at least one active group for the relevant preparation offers the advantage that the desired binding groups can directly be obtained. A group suitable for forming amino groups coupled to the carrier surface offers the advantage that such preparation carrier is in particular suitable for use in biotechnology, more in particular for binding amino acids.

In an alternative embodiment, a method according to the invention is characterized by the features of claim 4.

When the plastic used is not directly, or at least not sufficiently suitable for binding the relevant preparation, or at least cannot be transformed therefor by linkers, it is preferred that the carrier surface be treated in such a manner that on, or at least in the carrier surface, one or more active groups for the relevant preparation be provided, again in particular groups for forming amino groups by means of linkers, such as a —COOH or a —COO-methyl group. The advantage thus achieved is that as plastic for the carrier surface, a material can be used having particularly suitable properties therefor, such as, for instance, polyethene, while the treatment of the carrier surface provides that the formation of the amino groups is yet effectively enabled. In this respect, the advantage of plastic over, for instance, mica and glass, is that such treatment is possible in a particularly simple and effective manner, while in each case a suitable treatment can be selected, depending on the preparation to be bound. In particular —COOH groups actually also enable direct or indirect binding of, for instance, viruses and the like, while other active groups can also be provided, for instance —NH₂ groups.

In further elaboration, such method is preferably characterized by the features of claim 5.

By grafting the carrier surface with a plastic, a carrier surface that in itself binds insufficiently, if at all, can readily be treated for obtaining the desired activity. Especially the use of acrylic acid or methyl acrylate is particularly suitable therefor.

In a further advantageous embodiment, a method according to the invention is further characterized by the features of claim 6.

Surprisingly, it has been found that as the case may be, the surface roughness of a carrier surface can be further reduced by introducing —NH₂ groups in, or at least on the carrier surface. Thus, the surface roughness of a polyethene treated with acrylic acid or methyl acrylate can for instance be reduced thereby such that it can as yet be rendered suitable, or at least better suitable, for the desired use.

In further elaboration, a method according to the invention is further characterized by the features of claim 7, preferably by the features of claims 7 and 8.

By contacting a solution of a suitable monomer with the carrier surface and subsequently treating the plastic and solution, such that polymerization of at least a portion of the monomer occurs, a thin so-called adhesive layer can be provided on the carrier surface in a particularly simple manner, which adhesive layer is properly capable of effecting the desired bindings. By means of suitable irradiation, this polymerization can be effected and checked in a particularly effective manner.

Particularly suitable as carrier base are surfaces formed from, for instance, mica or glass, or materials having comparable surface roughness, hardness and/or porosity. In particular glass proves to be particularly suitable therefor.

Preferably, during use of a preparation carrier according to the present invention, a liquid is applied to the surface in a number of separate spots, each spot having a specific surface area. In each spot, one or more assays can be performed. By regulating the thickness of the adhesive layer, the size of each spot can be determined. Surprisingly, it has been found that with a relatively thin adhesive layer with a specific amount of liquid, a smaller spot is obtained than with the same amount of liquid with a thicker adhesive layer. Without wishing to be bound to any theory, this seems to result from the suction action of the adhesive layer, at least from deliquescence of the liquid which is greater with a relatively thick adhesive layer. By way of illustration, with an amount of liquid per spot of about 0.25 nl, with an adhesive layer having a thickness of from 1 to a few atoms, a spot can be obtained having a section of, for instance, 0.1 mm or less, while with an adhesive layer having a considerably greater thickness, spots can be obtained having a section of, for instance, 5 mm or more. These amounts and dimensions should not be construed as being limitative in any way.

With a method according to the invention, it is also possible to provide wells in a surface having the desired surface roughness through the use of, for instance, glass or mica bars having a spherical end that is pressed into the surface of the heated material, such as PE, preferably a matrix of such balls, pins or the like. As a result, each well is formed with an inner surface having said local low roughness. With such method, for instance wells having a volume of less than 3 μl, more in particular less than 1 μl, for instance 0.1 μl or less, can be obtained, into which drops of a particularly small volume can be deposited by means of jet printer technique or the like.

In a further elaboration, a method according to the invention is further characterized by the features of claim 10.

Coupling information-carrying polymers to the carrier surface offers the advantage that post-treatment of the surface is readily possible without the information-carrying polymers coming loose therefrom unintentionally, so that after said treatment, these polymers can readily be examined. If necessary, linkers can be used for the coupling of the polymers, whereby binding can be simplified, while the selectivity can be further increased for causing only the desired bindings to be effected or at least left over.

The invention further relates to preparation carrier, characterized by the features of claim 14.

Precisely a preparation carrier having a carrier surface manufactured from plastic, with a surface roughness such that markers of biochemical elements adhered thereto are perceptible and locatable thereon, offers the advantage that such preparation carrier is particularly simple to manufacture and adjust to the preparations to be examined, while such preparation carrier cam be used in a very simple manner, in particular also because it is relatively strong. The carrier surface being suitable for specific binding of the preparation, the advantage achieved is that during use, non-bound elements of the preparation can readily be washed away or treated otherwise, readily enabling all kinds of assays, known per se, to be performed on the preparation, such as ELISA. Precisely the specific binding of elements from the preparation to specific active groups of the carrier surface makes these assays possible. The particular flatness of the carrier surface offers the advantage that a particularly high information density can be obtained. The elements in the preparation that are to be examined can be positioned very close together without being distinguishable.

In further elaboration, a preparation carrier according to the invention is further characterized by the features of claim 17.

—COOH groups and —COO-methyl groups in or at least an the surface readily enable formation Of amino groups on the carrier surface by means of linkers, which groups are in particular suitable for coupling amino acids thereto. This offers the advantage that in a simple manner optionally presynthesized, complete or incomplete peptides, pieces of PNA, pieces of DNA, sugars, other organic molecules, proteins, viruses, bacteria and cells can be coupled to the surface, to the —COOH group, the —COO-methyl group or the formed amino group. For that matter, other active groups can be used as well. Thus, for instance bromoacetic acid can be synthesized on the carrier surface, to which peptides can subsequently be coupled via an SH-group of the peptides in

Hence, a preparation carrier according to the present invention offers the advantage that a great variety of possible chemical bindings of elements to the carrier surface can be obtained, as a result of which the preparation carrier is almost universally applicable.

The invention further relates to the use of microscopy and/or photography for biochemical research, characterized by the features of claim 19.

Precisely the use of a preparation carrier according to the present invention in cooperation with a microscope or a photo apparatus is advantageous, because the particular flatness of the carrier surface of the preparation carrier provides that in each case a proper focusing can be effected, so that particularly small color areas or other types of markers can readily be detected and distinguished from one another. Accordingly, in contrast with the known method, a particularly large number of markers can be distinguished on a relatively small surface, preferably involving the use of a confocal microscope scanner or a like microscope.

Printers, in particular a printer of the inkjet type, bubblejet type or comparable printers, operating by a drop-on-demand technique, such as for instance a printer having a glass capillary from which liquid is dropwise jetted in very small “drops” under the influence of a deformation of the wall by means of a piezoelectric element, offer the advantage that thus, in a relatively quick manner and with a high accuracy and reproducibility, small to particularly small amounts of slightly liquid preparation can be applied to a carrier surface in particularly closely spaced, distinct positions. If necessary, conjugates can thereby he added as well. In this manner, preparation carriers can simply and quickly be made ready for examination, while particularly much information can be applied to relatively small preparation carriers. This renders treatment and analysis of the information on the preparation carriers possible in a particularly simple manner.

The invention moreover relates to a microtiter plate or a like preparation carrier, comprising a matrix of wells, characterized by the features of claim 21.

Such preparation carrier is in particular suitable for use with a printer an described in claim 19. The advantage thus achieved in that the surface tension of the liquid to be introduced into the wells can be quickly and unequivocally introduced into the wells and the risk of air inclusion is prevented, Thus, for instance drops of a few tenths of μl or nl or less can be used. As a result, even less preparation and less surface are required. Preferably, yet not necessarily, the wells have an inner surface of a relatively low smoothness, obtained by a method according to any one of claims 1-13.

Preferably, such preparation carrier has outside dimensions of about 2.5 times 7.5 cm, allowing it to be placed in a standard detection apparatus, suitable for microscope slides.

Further exemplary embodiments of methods and preparation carriers according to the invention are given in the further subclaims.

To clarify the invention, exemplary embodiments of a method and a preparation carrier will hereinafter be specified with reference to the accompanying drawings. In these drawings:

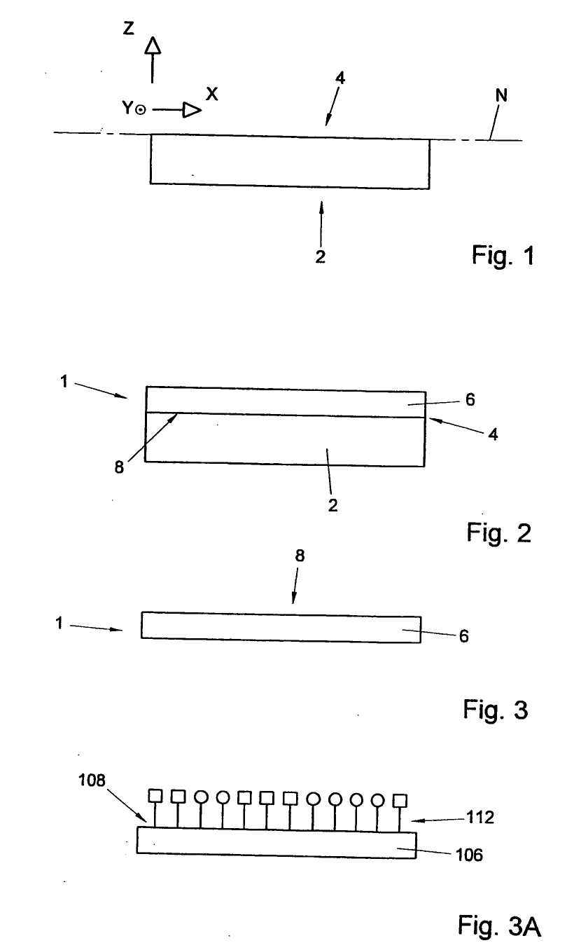

FIG. 1 shows a carrier base;

FIG. 2 shows a carrier base with a plastic layer applied thereto;

FIG. 3 shows the plastic layer removed from the carrier base;

FIG. 3a shows a plastic layer according to FIG. 3, in an alternative plastic;

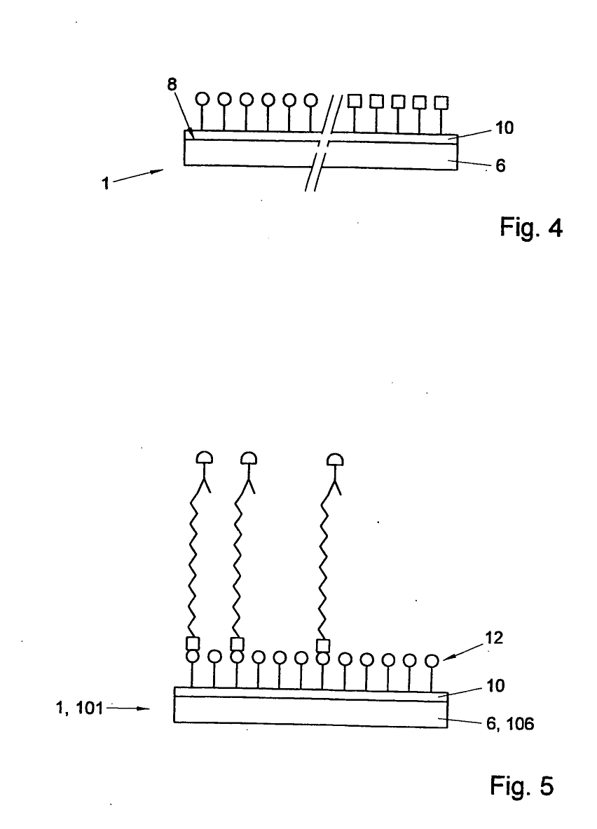

FIG. 4 shows the plastic layer with adhesive layer grafted on the carrier surface;

FIG. 5 is a schematic representation of a preparation carrier with peptides adhered to the carrier surface;

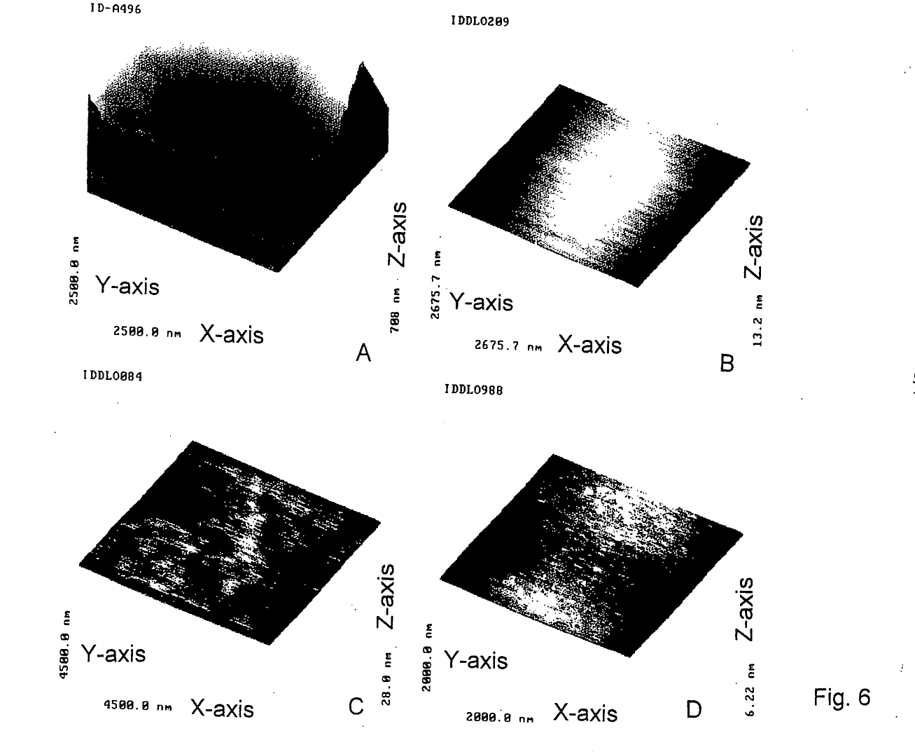

FIG. 6 is a much enlarged representation of, respectively, the surface of a customarily used pin, the surface of mica, the surface of a carrier surface according to present invention, manufactured from polyethene, and the surface of glass;

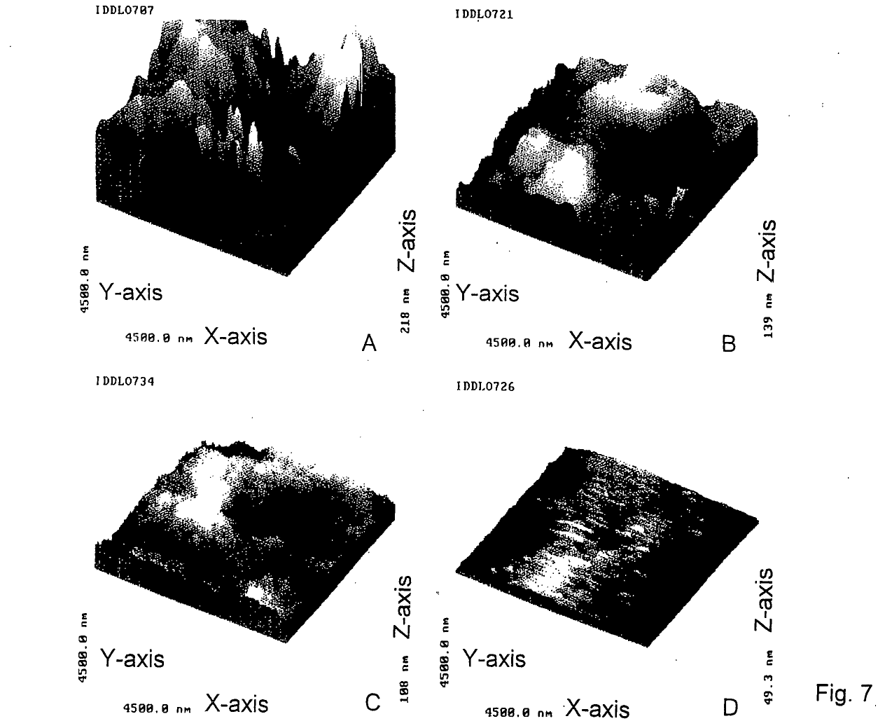

FIG. 7 shows four surfaces according to the present invention, with the carrier surface being grafted with a layer of methyl acrylate;

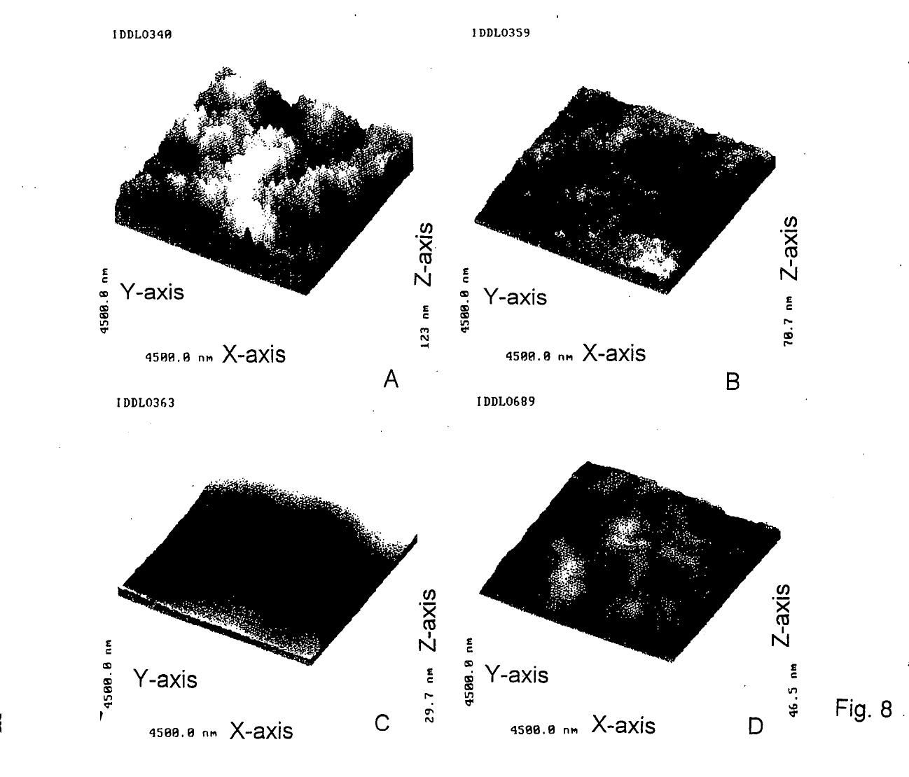

FIG. 8 shows four surfaces of a carrier surface according to the present invention, grafted with polyacrylate;

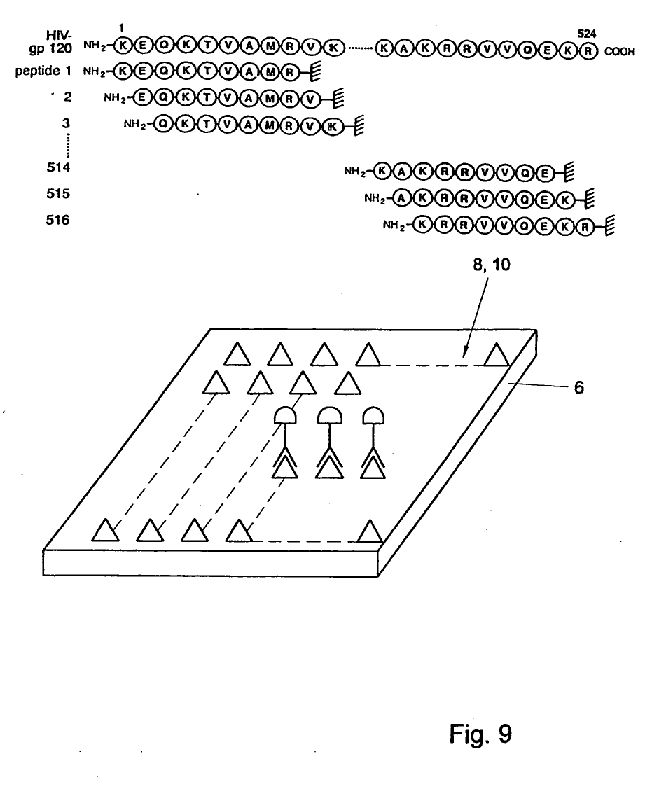

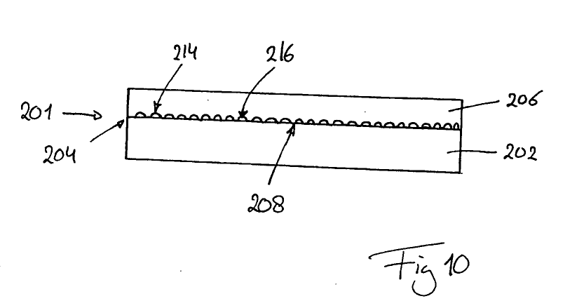

FIG. 9 is a schematic representation of a pepscan on a carrier surface; and

FIG. 10 shows a carrier base with a plastic layer applied thereto, comparable with FIG. 2, for the formation of a microtiter plate having a matrix of wells.

In this specification, identical or corresponding parts have identical or corresponding reference numerals. Further, as an example in this specification, unless otherwise indicated, a preparation carrier suitable for forming, on a carrier surface thereof, amino groups is started from, manufactured from treated polyethene or polypropene melted against glass. However, it will be understood that other plastics and another carrier base can be used as well, for instance a carrier base of mica and a polycarbonate, acrylic acid or methyl acrylate as plastic for the preparation carrier proper. In particular the last-mentioned plastics can offer the advantage that —COOH or —COO-methyl groups are directly available thereon. Polyethene and polypropylene are relatively inert. However, they offer the advantage of being relatively hard and strong without being brittle. Moreover, other plastics can readily be grafted thereon.

In this specification, in each case a relative flatness measure will be used, the maximal height (Z-axis) of projections above a nominal reference plane being given as percentage of one of the horizontal measures (X-axis) of the scanned surface. In this specification, this horizontal measure is in the order of magnitude of 2000-4500 nanometer. The measure for flatness V is therefore expressed in the following formula:

$$\frac{Z - axis}{X - axis} \times 100 \%$$

Examples of the flatness V of materials:

mica: V=0.1% (FIG. 6b);

glass: V=0.3% (FIG. 6d);

high-molecular polyethene: V=10% (FIG. 6a);

polyethene film: V=3% (FIG. 6b); and

a polyethene face formed according to the invention, V=0.6% (FIG. 6c);

polyethene pin surface: V≅28%.

These dimensions and values are given only as an example and should not be construed as being limitative in any way.

Legend: In the drawing:

□=—COOH or —COO-methyl

◯=—NH₂

[symbol] =antibody

[symbol] =peptide

∩=marker

FIG. 1 is a sectional side elevation of a carrier base 2, formed from mica, having a top surface 4 with a flatness V of about 0.1%. Hence, this means that on the face 4, there are unevennesses of a maximal height in the Z-direction measured above the nominal face N of at the most a few nanometers, for instance 4-5 nanometer. Hence, the surface 4 of mica is particularly flat. The surface 4 is for instance rectangular, with outer dimensions of 25×25 millimeter. The base carrier 2 has a thickness of, for instance, 0.5 millimeter.

In the condition shown in FIG. 2, a plastic layer 6 is provided on the smooth top surface 4 of the base carrier 2. In the embodiment shown, this is a polyethene film having an inherent smoothness of about 3%. The film layer has a thickness of, for instance, 0.035 millimeter.

The film layer 6 and/or the base carrier 2 are heated such that at least the side of the plastic layer 6 facing the surface 4 melts and deliquesces on the surface 4, after which the whole is cooled. Between the glass base carrier and the plastic layer 6, no adhesion of any significance will occur, allowing the plastic layer 6 to be readily removed from the base carrier 2 again. Surprisingly, it has been found that the surface 8 of the plastic layer 6 that faced the base carrier 2 has obtained a flatness V which is considerably better than the flatness V of the polyethene film used. The flatness of the carrier surface 8 is for instance about 0.6% when no further special measures are taken. It is further observed that, as the case may be, deliquescence of at least the part of the plastic layer 6 facing the base carrier 2 can also be effected, or at least partially effected, by for instance a chemical reaction.

FIG. 3 shows a preparation carrier 1 formed according to the present invention, with the carrier surface 8 facing upwards. In the embodiment shown, for instance polyethene or polypropene is used as plastic, which is relatively inert. As a result, binding thereto of biochemical elements is in fact not possible. FIG. 3A shows an alternative embodiment, wherein, as plastic layer 106, a plastic is used containing active groups 112, symbolically represented by spheres placed on rods. Such a plastic can for instance be a polycarbonate, an acrylic acid or methyl acrylate, in which for instance —COOH or —COO-methyl groups are present as active groups 112, in the drawing symbolically represented by, respectively, a square and a sphere on a rod.

FIG. 4 shows a preparation carrier 1 having a plastic layer 10 grafted thereon, for instance a polymerized layer of acrylic acid or methyl acrylate. Such layer 10 can be applied to the plastic carrier layer 6 of polyethene or another plastic as follows.

The plastic part 6 is immersed with its smooth carrier surface 8 in a solution of a monomer with a specific concentration, after which the solution with the plastic included therein is irradiated with radioactive radiation of a specific intensity, such that at least on the carrier surface 8 polymerization of the relevant monomer occurs.

Suitable monomer solutions are, for instance, a 0.6% or 6% acrylic acid (AC) monomer solution or a 0.6% or 6% methyl acrylate (MA) monomer solution. These solutions can for instance be irradiated with γ-radiation of, for instance, 2 or 12 kilo Gray (kGy). By a suitable choice of the irradiation time, a desired thickness of the relevant polymerized layer is thereby obtained on and partially in the carrier surface 8. Such adhesive layer has a thickness of for instance a few molecules or chains, so that the flatness of the carrier surface 8 is preserved as much as possible or even further increased.

FIGS. 7 and 8 show eight preparation carriers according to FIG. 4, grafted in solutions of, respectively, monomers methyl acrylate (FIG. 7) or acrylic acid (FIG. 8) with different concentrations and different irradiation amounts. As appears from FIG. 7, in particular the surfaces shown in FIGS. 7c, 7d and 7h are particularly flat and hence extremely suitable for preparation examination. The coding successively gives the carrier plastic (PE), the concentration of the solution (in %), the amount of irradiation (in kGy) and the grafting plastic (AC or MA) used. Of course, other combinations are also possible, for instance more or fewer or other monomers, other exposure amounts, other polymerization methods and other carrier plastics. Suitable choices therefrom are directly clear to anyone skilled in the art and can be determined without further invention.

A preparation carrier manufactured according to the invention can be utilized as follows.

By means of EDC(1-ethyl-3-(3-dimethylaminopropyl)carbodiamide) the peptide AC-SDSSFFSYGEIPFGK is applied to the carrier surface, coupled to an active group 12. Next, an ELISA is performed thereon with a monoclonal antibody (mAb) 59.7 ( 1/10,000) before and after disruption in an disrupting buffer. For this purpose, the carrier surface is cleaned ultrasonically at 70° in the presence of sodium dodecyl sulfate (SDS) and beta-mercaptoethanol (BME). The results of this ELISA are given in Table 1. It is clearly shown that on the carrier surface grafted with plastic (acrylic acid), the peptide is coupled, since after disruption, binding of the monoclonal antibody is still possible, while after disruption this is no longer possible at the bare carrier surface 8. It has been found that especially the grafted plastics (0.6/12Ac) and (0.6/2Ac) yield particularly satisfactory results.

Presynthetized complete peptides, as well as pieces of PNA, pieces of DNA, sugars or complete complex organic molecules, proteins, viruses, bacteria and cells can be coupled to a carrier surface of a preparation carrier according to the present invention. In principle, these can be coupled to the carrier surface as well as to amino groups formed on the carrier surface by linkers to the —COOH or —COO-methyl groups. Also, for instance bromoacetic acid can be coupled to an NH₂ group for obtaining a bromo group. To this bromo group, a peptide can be coupled via an SH group thereof. This may be advantageous in terms of price. A thus formed and treated preparation carrier can be observed with, for instance, a confocal microscope scanner. With this, a good view can be obtained of a relatively large surface, compared with for instance digitally stored comparison material.

In another application of a preparation carrier according to the present invention, viruses or antibodies are bound directly or via linkers with active groups 12 on or at least in the carrier surface 8.

The viruses or antibodies to be bound have or are provided with active groups, for instance —COOH groups and/or —NH₂ groups, which can be coupled directly or via linkers to the active groups 12 on or at least in the carrier surface 8, 10. Thus, for instance —NH₂ groups of a virus can be coupled to a —COOH group or an —NH₂ group of the carrier surface 8, 10, while —COOH groups of a virus can for instance be coupled to —NH₂ groups of the carrier surface 8, 10. As linkers, different chemicals can be used, for instance HMDA (Hexamethylenediamine) or EDA (Ethylenediamine). Thereby, for instance —NH₂ groups can be introduced as active groups in or on a carrier surface 8, 10 which only or substantially comprises for instance —COOH groups as active groups 12. HMDA can be used by coupling of Boc HMDA (Butyloxycarbonylhexamethylenediamine) via DCC (Dicyclohexylcarbodiimide) to the —COOH groups, whereby, after Boc-deprotection, —NH₂ groups become available for the coupling of antigen. When EDA is used, a surface 8, 10 treated with methyl acrylate can subsequently be treated with said EDA for, for instance, 72 hours at 40° C., with active —NH₂ groups becoming available. The first carrier surfaces are for instance PE(0.6/2Ac)-Hmda and PE(0.6/12Ac)-Hmda, while the second type of surface for instance meets PE(0.6/2MA)-EDA.

The other surfaces shown in FIGS. 7 and 8 are less flat. Introduction of —NH₂ groups into these surfaces, for instance in the manner described above, surprisingly leads to an improvement of the flatness V of these surfaces. This means that these surfaces, through the introduction of said —NH₂ groups therein, become also or at least even better suitable for use as preparation carrier for at least form-directed examination.

A further examination with a preparation carrier is globally described hereinbelow as an example and should not be construed as being limitative in any way.

FIG. 9 is a schematic representation of a pepscan examination, comprising the primary amino acid sequence of GP120 of HIV1, the main glycoprotein of HIV-1. Each circle represents an amino acid. For the amino acids, the single-letter code is used (A=alanine, C=cysteine, D=aspartic acid, E=glutamic acid, F=phenylalanine, G=glycine, H=histidine, I=isoleucine, K=lysine, L=leucine, M=methionine, N=asparagine, P=proline, Q=glutamine, R=arginine, S=Serine, T=threonine, V=valine, W=tryptophan, Y=tyrosine).

The amino acid sequence of GP120 of HIV-1 is divided into overlapping peptides as indicated. Peptide number 1 is the peptide starting with amino acid number 1 and ending with amino acid number 9, peptide number 2 is the peptide starting with amino acid number 2 and continuing to amino acid number 10, etc. The peptides are synthesized on the carrier surface, as shown in the lower part of FIG. 9. The peptides are indicated by individual triangles. Next, the complete carrier surface is brought into contact with the same antibody, represented by [symbol] . Some peptides will bind to this antibody. After the solution of antibody has been washed from the carrier surface, the antibody that is still present on the carrier surface and bound by the peptides can be demonstrated by means of anti-antibody conjugate. Thus, the sequence of the peptide that has bound to the antibody can directly be determined. Markers may be provided, preferably fluorescent markers, yet other markers may also be applied, for instance radioactive markers, precious metal such as gold, color markers and the like. As appears from FIG. 9, the individual peptides are particularly closely spaced. As the carrier surface is particularly flat, these peptides, at least the markers adhered thereto, can yet be detected individually with a confocal microscope scanner. This moreover means that only very little of the different elements needed for the assay is necessary, such as the peptides to be distinguished, conjugate, antibody, anti-antibody conjugate and the like.

After the desired sequence of the or each relevant peptide has been established, the antibody can be removed from the peptides and the peptides can be reused. Through the use of a preparation carrier according to the present invention, particularly many different peptides can be synthesized in a relatively short time.

It is preferred that the peptides be applied to the carrier surface by means of an inkjet printer or a bubblejet printer or like printers that are based on the drop-on-demand technique, because this enables a particularly dense packing of the relevant peptides on the carrier surface in a simple, quick manner and with great precision and reproducibility. For instance, “drops” of from 0.25 to 0.5 nanoliter can be jetted at 1 to 2 kilohertz. The carrier plastic has the advantage of being properly resistant to the peptide chemistry, which seems to be too aggressive if glass were used as carrier. With a method according to the present invention, a very drastic microturization of the pepscan can be obtained. For scanning the surface with peptides and the like bound thereto, a confocal microscope is preferably used. Precisely with such a microscope, the particular smoothness of the surface has great advantages.

Table 2 shows for the eight surfaces shown in FIGS. 7 and 8 ELISA values of monoclonal antibodies and their associated peptides, synthesized on the relevant carrier surfaces. This demonstrates that synthesization is possible on all grafted surfaces used, regardless of the thickness thereof. Thus, peptides, DNA, PNA and like information-carrying polymers can be synthesized thereon.

A preparation carrier according to the present invention offers as important advantage over the prior art that in a particularly simple manner, different types of active groups can be provided on, or at least in the carrier surface, such as the —COOH groups and —NH₂ groups mentioned. According to the desired application and the desired bindings, the carrier surface can be treated in a suitable manner, if necessary. Moreover, the active groups can be provided so as to be particularly close together, so that a high density of the elements to be detected from the preparation can be obtained, for instance 999 peptides per cm². Accordingly, the resolving power of the detection technique used can be increased considerably, or at least be utilized in a more optimal manner.

The flatness of the carrier surface 8 can possibly be further increased through the use of appropriate techniques, for instance vacuum techniques for placing and melting the plastic layer 6 on the carrier base 2, or at least causing it to deliquesce thereon. This prevents gas inclusions from possibly leading to unevennesses.

FIG. 10 is a sectional side elevation of a carrier base 202 having a top surface 204, on which protrusions 214 are provided, which are substantially spherical, for instance hemispheres. The convex side thereof faces away from the carrier base 202. A plastic layer 206 is provided over the base carrier 202 and the protrusions 214, for instance as described with reference to FIGS. 1 and 2. As a result, cavities 216 are obtained in the plastic layer 206, which cavities have an inner surface corresponding to the outer shape of the protrusions 214 and a surface roughness comparable therewith. The protrusions 214 can for instance be formed by glass or mica parts, such as balls pressed approximately halfway into the base carrier 202. They may also be formed integrally therewith. Thus, wells 216 are obtained, having an inner surface of a particularly low surface roughness, for instance in the order of magnitude as described with reference to FIGS. 1-9. The wells are preferably arranged in a N×M matrix, comparable with known microtiter plates.

The wells 216 may have a volume corresponding to that of the wells of known microtiter plates, i.e. in the order of magnitude of, for instance, about 3 μl. However, it is also possible to make them of a considerably smaller design, for instance with a diameter such that wells 216 are obtained having a volume which is considerably less than 3 μl, for instance less than 1 μl or even less than 0.1 μl. These wells are preferably, yet not necessarily, formed with protrusions 214 having a particularly smooth outer surface. A carrier 206 having such particularly small wells 216 offers the advantage that very little preparation is necessary and a great many wells 216 can be provided on a relatively small surface. Such preparation carrier 201 is in particular suitable for use with a printer of the drop-on-demand type, such as an inkjet or bubblejet printer or the like. Thus, particularly small volumes can be introduced into the well 216 without involving air inclusion in the well, while the surface tension of the preparation liquid to be introduced can be overcome relatively easily.

In an alternative embodiment, not shown, instead of protrusions, pins are used whose ends correspond to the protrusions 214, which pins are moved relative to the plastic layer 206 for forming the desired cavities 216. Also, in this manner, regular or other patterns of wells 216 can be obtained of the desired volume. Wells 216 of said relatively small volume (less than 3 μl, in particular less than 1 and preferably less than 0.1 μl) are in particular suitable for analysis of preparations included therein, by means of for instance luminescence, fluorescence or comparable markers which can be detected without utilizing HFM microscopy.

The invention is in no way limited to the exemplary embodiments shown in the drawing and specification. Many variations thereto are possible within the framework of the invention outlined by the appended claims.

For instance, other plastics may be used for forming the carrier surface and/or for grafting the layer 10 thereon. Suitable plastics may for instance be selected on the basis of the desired active groups, the desired hardness or flexibility, the desired combination of carrier plastic and grafting plastic, possible resistance to, for instance, chemicals, irradiation, exposure and the like. Such choices will be readily understood by anyone skilled in the art within the framework of the invention.

Further, preparation carriers according to the present invention may also be used for other examinations, for instance examinations involving the use of markers for establishing the presence of specific elements, for instance fluorescent, coloring or radiant markers. In the exemplary embodiments shown, the plastic layer is in each case provided on the base carrier, yet it is of course also possible to process a plastic layer with a sufficiently smooth surface of a base carrier that is moved against or along the surface of the plastic layer, for instance a base carrier of mica or glass. It is also possible to cause polymerization of a plastic to take place on a base carrier having the desired smoothness or to effect the formation of plastic having suitable properties thereon in a different manner. The carrier may for instance be a portion of a mold. Of course, all kinds of different preparations may be bound on a preparation carrier according to the present invention. The viruses described only serve as example.

These and many comparable variations are understood to fall within the framework of the invention outlined by the claims.

TABLE 1
OD405

| | | |
|---|---|---|
| Surface only flattened | | |
| base polymer | Flat 0.6/2AC | Flat 0.6/12AC |

| (1) | (2) | (1) | (2) | (1) | (2) |
|---|---|---|---|---|---|
| 3231 | 192 | 3502 | 1517 | 3127 | 2754 |

TABLE 2
OD405

| graft type | peptide AcGQPAVRNE | peptide AcSFFSYGEI |
|---|---|---|
| substrate polymer | MAB 3C8 1/2000000 | MAB 57.9 1/750000 |
| 6/12MA | 950 | 590 |
| 6/2 MA | 857 | 681 |
| 0.6/12MA | 311 | 547 |
| 0.6/2MA | 508 | 312 |
| 6/12AC | 977 | 264 |
| 6/2AC | 862 | 286 |
| 0.6/12AC | 1178 | 875 |
| 0.6/2AC | 939 | 1135 |

Especially grafts 0.6/12AC and 0.6/2AC yield good results.

1. A method for manufacturing a preparation carrier, wherein:
on at least one surface of a carrier base, a layer of polyethene or polypropene is provided, wherein the carrier base is a surface formed from mica or glass or a material having a flatness V of about 0.1%-0.3%,
wherein the plastic layer is treated thermally and/or chemically, such that the surface roughness of the side of the plastic that faces the carrier base is reduced such that the flatness V is less than 3%, while the plastic does not adhere to the carrier base,
whereupon the plastic is removed from the carrier base, with the released surface of the plastic forming a carrier surface,
wherein the flatness V is expressed in the formula Z-axis/X-axis×100% with the X-axis being between 2000-4500 nanometer.

2. A method according to claim 1, wherein the plastic is provided over the at least one relevant face of the carrier base by melting said plastic at least partially.

3. A method according to claim 1, wherein as plastic, a monomer or polymer is used having at least one active group for the relevant preparation, in particular a group that can be used for forming an amino group.

4. A method according to claim 1, wherein the carrier surface is treated such that the carrier surface comprises at least one active group for the relevant preparation, in particular a group that can be used for forming an amino group.

5. A method according to claim 4, wherein the carrier surface is grafted with a plastic, in particular by means of a monomer or polymer, preferably acrylic acid or methyl acrylate.

6. A method according to claim 4, wherein by introduction of —NH₂, groups in, or at least on the carrier surface, the surface roughness thereof is reduced.

7. A method according to claim 4, wherein at least the plastic layer on at least the carrier surface is brought into contact with a solution of a monomer, whereupon the plastic and the solution are treated such that polymerization of at least a portion of the monomer occurs on the carrier surface, for which purpose, preferably, the plastic together with the solution is exposed to radiation.

8. A method according to claim 3, wherein the active groups are converted into amino groups by means of linkers.

9. A method according to claim 3, wherein information-carrying polymers are coupled or synthesized to at least a number of active groups, optionally through the agency of suitable linkers.

10. A method according to claim 1, wherein the carrier surface is formed by or comprises at least one substantially spherical body having a diameter such that in the plastic, on the side facing the carrier base, at least one well is obtained having a volume of less than 3 μl.

11. A method according to claim 3 wherein the active group is a —COOH or a —COO-methyl group.

12. A method according to claim 4 wherein the active group is a —COOH or a —COO-methyl group.

13. A method according to claim 10, wherein said well has a volume of less than 1 μl.

14. A method according to claim 10, wherein said well has a volume of less than 0.1 μl.

15. A method according to claim 10 wherein in the plastic, on the side facing the carrier, a matrix of wells is obtained wherein the wells have a volume of less than 3 μl.

16. A method according to claim 10 wherein in the plastic, on the side facing the carrier, a matrix of wells is obtained wherein the wells have a volume of less than 1 μl.

17. A method according to claim 10 wherein in the plastic, on the side facing the carrier, a matrix of wells is obtained wherein the wells have a volume of less than 0.1 μl.

18. A method according to claim 1 wherein a carrier base is used of which at least said face is manufactured from glass.

19. A method according to claim 1, wherein the surface roughness of the side of the plastic that faces the carrier base is reduced such that the flatness V is between 0.1% and 0.6%.
